(19) United States
(12) Patent Application Publication
(10) Pub. No.: US 2005/0288343 A1
(43) Pub. Date: Dec. 29, 2005

(54) Process of preparing substituted carbamates and intermediates thereof

(76) Inventors: Andrew Rusowicz, Manville, NJ (US); Gregory C. Lane, Yardley, PA (US); Manohar Saindane, Monmouth Junction, NJ (US); Hyei-Jha Chung, Plainsboro, NJ (US); Mary F. Malley, Lawrenceville, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/130,048

(22) Filed: May 16, 2005

Related U.S. Application Data
(60) Provisional application No. 60/572,397, filed on May 19, 2004.

(51) Int. Cl. A61K 31/421; C07D 263/34

(52) U.S. Cl. 514/374; 548/236

(57) ABSTRACT

An improved process of preparing substituted carbamate derivatives, and crystalline forms thereof, useful for the treatment of dyslipidemia and diabetes, and intermediates thereof are provided.

### REFERENCE TO OTHER APPLICATIONS

The present application takes priority from U.S. provisional application No. 60/572,397 filed May 19, 2004, the disclosure of which is incorporated herein by reference.

### BACKGROUND OF THE INVENTION

The present invention relates to preparation of substituted carbamate derivatives, such as N-[(4-methoxyphenoxy)carbonyl]-N-[[4-[2-(5-methyl-2-phenyl-4-oxazolyl)ethoxy]phenyl]methyl]glycine (also referred to as muraglitazar) and crystalline forms thereof. The substituted carbamate derivatives of the present invention are useful for the treatment of dyslipidemia, diabetes, and atherosclerosis, and intermediates thereof. In particular, the process of the invention includes an improved procedure for synthesizing and isolating the final product in a single step.

### BACKGROUND OF THE INVENTION

U.S. Pat. No. 6,414,002 discloses a class of substituted carbamate acid derivatives useful for treating a range of symptoms, disorders and diseases. Among these compounds is ((4-methoxy-phenoxycarbonyl)-{4-[2-(5-methyl-2-phenyl-oxazol-4-yl)-ethoxy]-benzyl}-amino)-acetic acid (also referred to as muraglitazar), which is a pharmaceutically active compound that has been shown to exhibit pharmacological activity including modulation of blood glucose levels, triglyceride levels, insulin levels and non-esterified fatty acid (NEFA) levels in warm-blooded animals including humans. The compound activates peroxisome proliferator-activated receptors (PPAR) α (insulin sensitizer) and γ (lipids/cholesterol lowering), and thus may be useful for the treatment of dyslipidemia, atherosclerosis and diabetes, especially Type 2 diabetes.

U.S. Patent Application Ser. No. 60/556,331, filed Mar. 25, 2004 describes various tablet formulations and methods of preparation thereof for certain other substituted carbamate derivatives usable for the treatments as described herein.

It would be a significant advance in the art to provide processes of preparing ((4-methoxy-phenoxycarbonyl)-{4-[2-(5-methyl-2-phenyl-oxazol-4-yl)-ethoxy]-benzyl}-amino)-acetic acid and related substituted carbamate derivatives as described in Application Ser. No. 60/556,331 having improved product yields, purity and ease of production over the prior art processes. Such objectives are met by various embodiments of the presently claimed invention.

### SUMMARY OF THE INVENTION

The present invention is generally directed to a process of preparing the compound of Formula (I) and intermediates thereof, 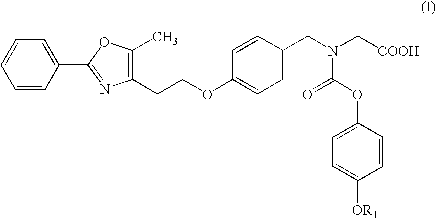
wherein R₁ is selected from alkyl, aryl, alkenyl, alkynyl, alkyloxy(halo)aryl, alkyl(halo)aryl or cycloalkylaryl, preferably alkyl, and more preferably methyl. Exemplary embodiments of this invention are directed to the compound of formula (Ia): 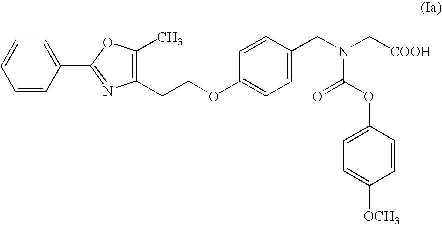
(also referred hereinafter as “((4-methoxy-phenoxycarbonyl)-{4-[2-(5-methyl-2-phenyl-oxazol-4-yl)-ethoxy]-benzyl}-amino)-acetic acid”) or “N-[(4-methoxyphenoxy)carbonyl]-N-[[4-[2-(5-methyl-2-phenyl-4-oxazolyl)ethoxy]phenyl]methyl]glycine” or “muraglitazar”), and to intermediates thereof.

Further embodiments of the invention relate to particular physical crystalline forms of N-[(4-methoxyphenoxy)carbonyl]-N-[[4-[2-(5-methyl-2-phenyl-4-oxazolyl)ethoxy]phenyl]methyl]glycine.

The process of the present invention is characterized by significant reduction in contaminants and by-products during synthesis, whereby the yield and purity of the desired end product is enhanced. The advantages of the present invention further enable the process to be implemented at least substantially in a single vessel, thus enabling further reduction in the cost and/or time of production. The process of the present invention provides direct isolation of the final product from a crude saponified reaction mixture, and forgoes the isolation of an intermediate product. The final product is crystallized from an aqueous mixture containing minimal by-product salts.

In accordance with one aspect of the present invention, there is provided a process of preparing the compound of Formula (I) which includes the steps of:

reacting an acid salt of the compound of Formula (II) 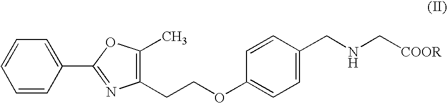
wherein R is alkyl,

with the compound of Formula (III) 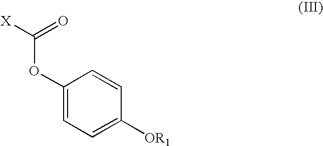
wherein X is selected from Cl, Br or I, and R₁ is selected from alkyl, aryl, alkenyl, alkynyl, alkyloxy(halo)aryl, alkyl(halo)aryl or cycloalkylaryl, to yield the compound of Formula (IV), and

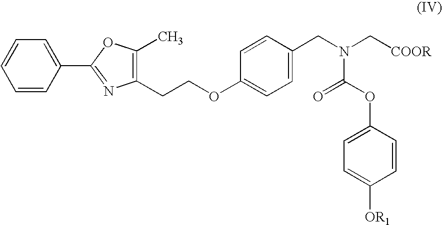

hydrolyzing the compound of Formula (IV) to yield the compound of Formula (I).

In another aspect of the present invention, there is provided a process of preparing stable acid salts of the compound of Formula (II) as intermediate products useful for the synthesis of the desired final product. The acid salt intermediate product affords improved yield and purity of the final product, and also provides added flexibility in scheduling production runs and enables different stages of the reaction to be carried out at different manufacturing facilities.

In another aspect of the invention there is provided a process of preparing an acid salt of the compound of Formula (II), wherein the process includes the steps of:

reacting the compound of Formula (V)

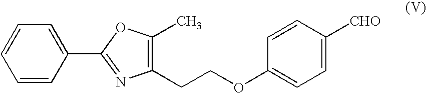

with a glycine methyl ester acid salt (e.g., hydrochloride salt) to form a Schiff base

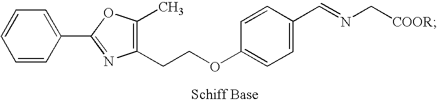

catalytically reducing the Schiff base to yield the compound of Formula (II)

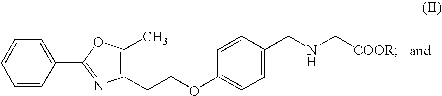

treating the compound of Formula (II) with an acid salt-forming reagent (e.g., alkylchlorosilane such as trimethylchlorosilane and in the presence of an alcohol such as methanol to produce a corresponding HCl salt) to yield the acid salt of the compound of Formula (II).

In another aspect of the present invention, there is provided a process of preparing the compound of Formula (IV), comprising reacting an acid salt of the compound of Formula (II) with the compound of Formula (III) in the presence of a base.

Suitably, the process of the invention may be carried out in single or multiple batches, or may be run as a continuous in-line operation. Moreover, the process may be telescoped to provide the steps in the formation of acid salt of the compound of Formula (II) to be used directly in the formation of the compound of Formula (IV) and for the subsequent hydrolysis to form the desired free acid product in a single reaction vessel. Such an in situ or one-pot process desirably improves the efficiency of manufacturing the product on a large scale, while maintaining satisfactory levels of product purity and yield.

The present method of preparation of the acid salt may precede the one pot reaction described above, however preparation of this reagent may also be done in a single reaction vessel in which the subsequent carbamate formation and hydrolysis steps are performed.

The reaction product may be extracted, isolated and purified by any conventionally acceptable means, and the desired product of Formula I obtained as the free acid or as a salt, solvate, ester, prodrug or isomer thereof, as may be desirable for the intended route of administration. The solid product, whether recovered in crystalline or amorphous form, may be milled to provide particles of the desired size. In certain embodiments of the present invention, the recovered product may for example be sonicated to provide particles of a desired size. The desired particle size will vary according to the intended use.

### BRIEF DESCRIPTION OF THE FIGURES

The invention is illustrated by reference to the accompanying drawings described below.

FIG. 1 shows calculated (simulated at 22° C.) and observed (experimental at room temperature) powder X-ray diffraction patterns of the N-1 crystalline form of the compound of Formula Ia.

FIG. 2 shows ¹³C NMR CPMAS spectrum for the N-1 crystalline form of the compound of Formula Ia.

FIG. 3 shows thermogravimetric analysis curve of the N-1 crystalline form of the compound of Formula Ia.

FIG. 4 shows differential scanning calorimetry thermogram of the N-1 crystalline form of the compound of Formula Ia.

FIG. 5 shows moisture-sorption isotherm analysis of the N-1 crystalline form of the compound of Formula Ia.

### DETAILED DESCRIPTION OF THE INVENTION

The present invention is generally directed to the preparation of the compound of Formula (I) and salts, solvates, esters, prodrugs or isomers thereof 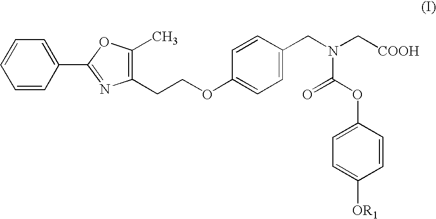
wherein R₁ is selected from alkyl, aryl, alkenyl, alkynyl, alkyloxy(halo)aryl, alkyl(halo)aryl or cycloalkylaryl, which includes the reaction of an acid salt of the compound of Formula (II) 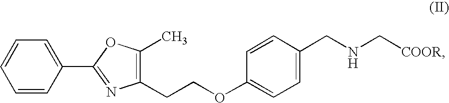
wherein R is alkyl, with the compound of Formula (III) in the presence of a base followed by hydrolysis of the resulting product, (i.e., the compound of Formula (IV))

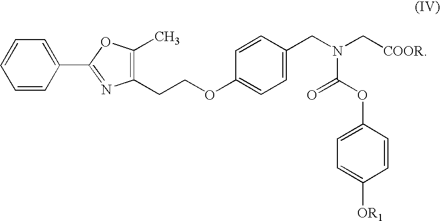

Employment of an acid salt of the compound of Formula (II), preferably in the form of a hydrochloride salt, significantly reduces or eliminates typical undesirable by-products to provide a product with improved yield and purity and better isolation, and facilitates the production of the desired end product in a one pot system (i.e., in a single reaction vessel). This permits the intermediary carbamate ester (e.g. a compound of Formula (IV)) to be hydrolyzed without requiring isolation, thus affording the one pot process to be desirably realized.

As used herein, the term “alkyl,” as employed herein alone or as part of another group, refers to both straight and branched chain hydrocarbons containing 1 to 20 carbon atoms, preferably 1 to 10 carbon atoms, more preferably 1 to 8 carbon atoms in the normal chain. The term “alkenyl,” as employed herein alone or as part of another group, refers to both straight and branched chain hydrocarbons containing 2 to 20 carbon atoms, preferably 2 to 12 carbon atoms, more preferably 2 to 8 carbon atoms in the normal chain, which include one to six double bonds in the normal chain. The term “alkynyl,” as employed herein alone or as part of another group, refers to both straight and branched chain hydrocarbons containing 2 to 20 carbon atoms, preferably 2 to 12 carbon atoms, more preferably 2 to 8 carbon atoms in the normal chain, which include one to three triple bonds in the normal chain. The alkyl, alkenyl and alkynyl substituents may further include an oxygen or nitrogen atom positioned terminally or within the normal chain, and/or may be further substituted by 1 to 4 substituent groups such as F, Br, Cl, I or CF₃, alkoxy, aryl or cycloalkyl. The term “cycloalkyl,” as employed herein alone or as part of another group, refers to saturated or partially unsaturated cyclic hydrocarbons containing 1 to 3 rings and containing a total of 3 to 20 carbon atoms, any of which may be substituted or unsubstituted. The term “aryl,” as employed herein alone or as part of another group, refers to monocyclic and bicyclic aromatic groups containing 6 to 10 carbon atoms in the ring portion, and may optionally be substituted at available carbon atoms.

The term “acid salt of the compound of Formula (II)” refers to any suitable conventional acid-addition salt of the compound of Formula (II) (i.e., {4-[2-(5-methyl-2-phenyl-oxazol-4-yl)-ethoxy]-benzylamino}-acetic acid methyl ester) that retains the pharmacological properties of the compound of Formula (II) and is formed from suitable organic or inorganic acids and can be readily converted to the compound of Formula (II) in relatively high yields. Representative acid-addition salts of the compound of Formula (II) include those derived from the reaction of alkylchlorosilanes such as trimethylchlorosilane with an alcohol, such as methanol, to generate hydrochloric acid in situ, those derived from inorganic acids such as hydrochloric acid, hydrobromic acid, hydroiodic acid, phosphoric acid, bromic acid, sulfuric acid and nitric acid, and those derived from organic acids such as acetic acid, citric acid, fumaric acid, lactic acid, malic acid, methanesulfonic acid, salicylic acid, succinic acid, tartaric acid, and the like. A preferred acid salt of the compound of Formula (II) is the hydrochloride salt.

The acid salt of the compound of Formula (II) may be advantageously prepared from the compound of Formula (V), which is itself prepared by the known synthesis procedures such those disclosed, for example, in European Patent Application No. EP177355 and PCT World Patent Application No. WO 99/5027, the disclosures of which are herein incorporated by reference. The employment of a compound of Formula (V) as a starting material for the production of an exemplary acid salt of the compound of Formula (II) is shown in the processes illustrated in Reaction Schemes 1 and 2 below (which are preferred). As previously discussed, the process of the present invention may be advantageously carried out in a single vessel.

It will be understood that where typical or preferred process conditions (i.e. reaction temperatures, times, mole ratios of reactants, solvents, pressures, etc.) are given, other process conditions may also be used unless otherwise stated. Exemplary reagents and procedures for these reactions appear hereinafter and in the working Examples. Protection and deprotection in the Reactions Schemes below may be carried out by procedures generally known in the art (see, for example, Greene, T. W. and Wuts, P. G. M., Protecting Groups in Organic Synthesis, 3^{rd} Edition, 1999 [Wiley]). Optimum reaction conditions may vary with the particular reactants or solvents used, however such reaction conditions may be determined by one of ordinary skill in the art through routine optimization procedures.

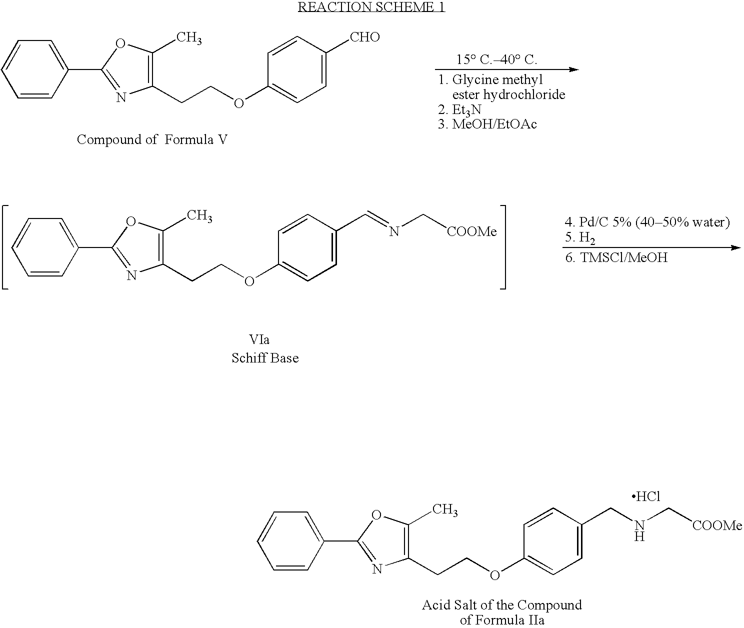

The preparation of an intermediate acid salt of the compound of Formula (IIa) (i.e., hydrochloride salt) is illustrated in Reaction Scheme 1. The compound of Formula (V) is treated with a glycine methyl or ethyl ester acid salt (such as the hydrochloride salts, hydrobromide salts, hydrosulfate salts, hydroiodide salts, phosphate salts, sulfate salts, nitrate salts, bromide salts, and the like, and combinations thereof) in the presence of an organic base, preferably an amine such as triethylamine and an organic solvent such as methanol and ethyl acetate or methyl tert-butyl ether to yield the intermediary Schiff base (VIa). The Schiff base can be reduced in accordance with procedures known in the literature (e.g. Abdel-Magid et al., J. Org. Chem. 1996, 61, 3849). Alternatively, the reduction of the Schiff base may be carried out by other suitable reducing agents selected from metal hydrides such as alkali metal boranes (e.g., NaBH₄ and LiBH₄). The reduction reaction is preferably carried out at a temperature of about 15° C. to 40° C.

The Schiff base, as discussed above, is catalytically reduced in the presence a suitable catalyst such as 5% Pd/C or Pt/C and hydrogen gas under standard conditions to produce an amine. In a preferred embodiment, the catalyst is suitably removed by filtration or the like. Preferably, the resulting filtrate is diluted with water and brine, and extracted with ethyl acetate or methyl tert-butyl ether (MTBE). The rich organic phase is then washed with water. The rich organic phase is then distilled to azetropically remove residual water. The resulting amine is treated with an acid salt-forming reagent including alkylchlorosilane such as trimethylchlorosilane in the presence of an alcohol such as methanol, to yield a desired intermediate product in the form of a hydrochloride acid salt of the compound of Formula (IIa). The optimal addition rate for the acid salt-forming reagent such as trimethylchlorosilane is preferably via a cubic addition profile, which maximizes removal of organic contaminants and optimizes particle size for ease of filtration.

Alternative acid salt-forming reagents may include those capable of producing acid salts of the compound of Formula (II) including organic and inorganic acids such as hydrochloric acid, hydrobromic acid, hydrosulfuric acid, and other suitable organic and inorganic acid salt-forming reagents as known to one skilled in the art.

The intermediate product, the acid salt of the compound of Formula (II), is used in the preparation of the final product and results in desirable purity and yield profiles. The acid salt of the compound of Formula (II) also provides added flexibility to the overall process as the process steps leading up to the preparation of the compound of Formula (II) can be performed separately from the process steps for the conversion to the desired final product as will be described below.

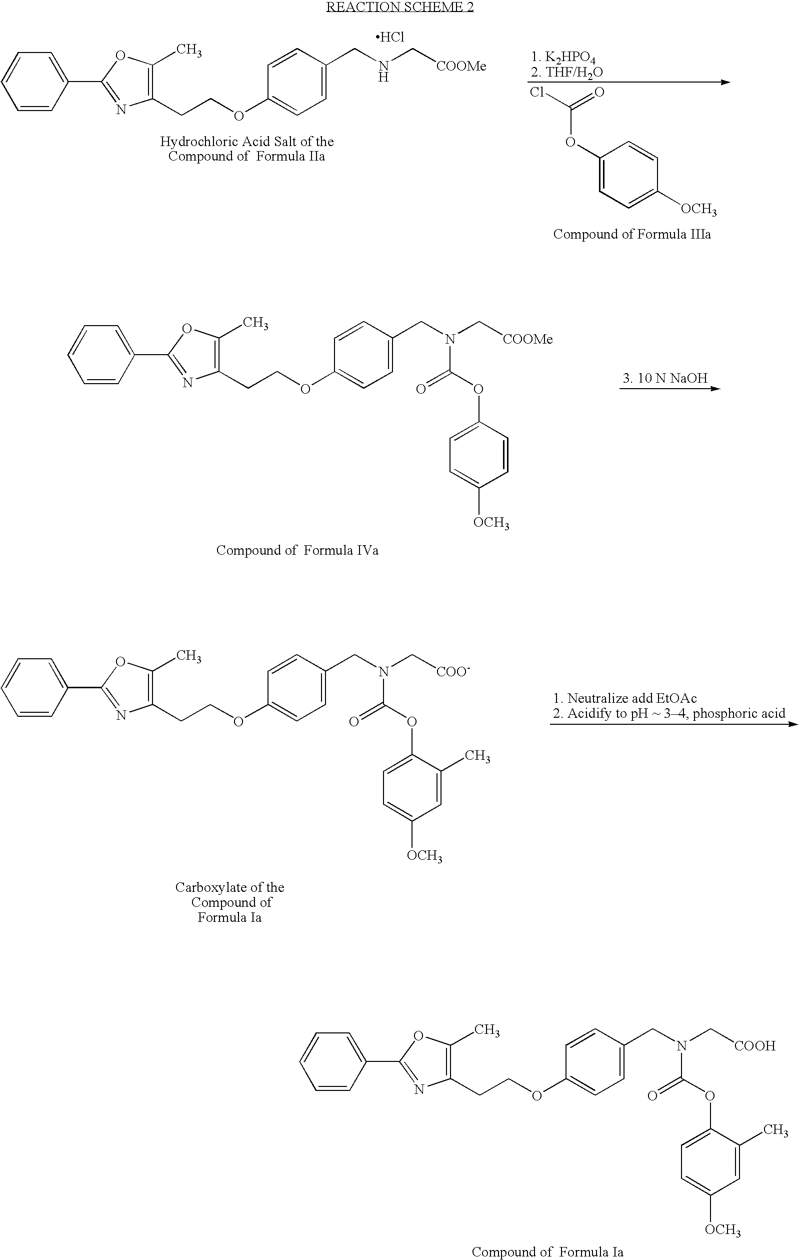

To prepare the compound of Formula (Ia), the intermediate product, acid salt of the compound of Formula (IIa), is treated with a base such as alkali metal hydroxides or mixed alkali carbonates, preferably an aqueous phosphate buffer such as dibasic potassium phosphate, in the presence of water and an organic solvent such as tetrahydrofuran to yield a free amine. The solvent mixture of water and tetrahydrofuran is preferred for its low cost, easy disposal and good safety profile. The free amine is treated with a chloroformate such as methoxyphenyl choroformate under standard conditions known in the art to yield an intermediate carbamate ester, the compound of Formula (IVa). Preferably, the reaction is carried out at a temperature of about 25° C. to 40° C. The intermediary carbamate ester (i.e., compound of Formula (IVa)) remains in the presence of impurities and by-products including, for example, K₂HPO₄, KH₂PO₄, KCl and the like, in the reaction mixture for subsequent hydrolysis.

Thereafter, the ester group of the compound of Formula (IVa) is converted to a free acid through hydrolysis with a suitable base such as alkali metal hydroxides, preferably sodium hydroxide, preferably at a pH of at least 14, to produce a carboxylate of the compound of Formula (I). Preferably, the sodium hydroxide base is employed at a concentration of about 10N. The reaction is preferably carried out at a temperature of about 30° C. to 60° C.

The reaction mixture is neutralized with a suitable acid such as phosphoric acid, then diluted with ethanol and thereafter phase-separated. The organic phase is diluted with water and preferably ethanol and further acidified with a suitable acid such as phosphoric acid, preferably to a pH of less than 3.5, to produce the compound of Formula (I) in crystal form. Phosphoric acid is preferred because it significantly minimizes ethyl ester impurities. The product slurry is diluted with water to facilitate removal of by-products containing inorganic salts prior to isolation.

In an alternate embodiment of the present invention, the final product may be extracted by adding a crystallizing agent selected from organic solvents such as, for example, n-heptane or ethyl acetate, or organic alcohols such as, for example, ethanol to induce controlled crystallization to yield the final desired product that is at least substantially crystalline. This crystallization process significantly minimizes the formation of undesirable amorphous precipitates, typically observed when evaporation is used to concentrate the reaction mixture. It is highly desirable to produce a substantially crystalline form of the product especially for use in pharmaceutical dosage forms. The final product is then filtered and dried. Optionally, the filtered and dried final product may thereafter be dissolved in an alcohol such as ethanol and subjected to recrystallization to optimize bulk removal of contaminants and thus improve purity and yield.

The compound of Formula (Ia) has been observed to modulate blood glucose levels, triglyceride levels, insulin levels and non-esterified fatty acid (NEFA) levels in warm-blooded animals including humans. Moreover, as demonstrated in U.S. Pat. No. 6,414,002, the compounds of Formula (I) and pharmaceutically acceptable salts, solvates, esters, prodrugs and isomers thereof may generally be useful in treating dyslipidemia, hyperglycemia, hyperinsulinemia, hyperlipidemia, atheroschlerosis and diabetes including Type 2 diabetes, and related diseases. For therapeutic use, the compounds of Formula (I) may be administered as a pharmaceutical composition which includes a solid or liquid pharmaceutically acceptable carrier and, optionally, pharmaceutically acceptable adjuvants and excipients.

The pharmaceutical compositions include suitable dosage forms for oral, parenteral (including subcutaneous, intramuscular, intradermal and intravenous) bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques.

If a liquid carrier is employed, the preparation may be in the form of a syrup, emulsion, soft gelatin capsule, sterile solution for injection, an aqueous or non-aqueous liquid suspension, or may be a dry product for reconstitution with water or other suitable vehicle before use. Liquid preparations may contain conventional additives such as sweeteners, suspending agents, emulsifying agents, wetting agents, non-aqueous vehicle (including edible oils), preservatives, as well as flavoring and/or coloring agents.

For parenteral administration, a vehicle normally will principally comprise sterile water, although saline solutions, glucose solutions and like may be utilized. Injectable suspensions also may be used which may require conventional suspending agents. Conventional preservatives, buffering agents and the like also may be added to the parenteral dosage forms. Particularly useful is the administration of the compounds of Formula (I) directly in parenteral or oral formulations. The pharmaceutical compositions are prepared by conventional techniques appropriate to the desired preparation containing appropriate amounts of the active ingredient, that is, the compound of Formula (I) according to the invention. See, for example, Remington's Pharmaceutical Sciences, Mack Publishing Company, Easton, Pa., 17th edition, 1985.

The dosage of the compounds of Formula (I) to achieve a therapeutic effect will depend on factors including, but not limited to, the age, weight and sex of the patient and mode of administration, and the particular disorder or disease concerned. It is also contemplated that the treatment and dosage of the particular compound may be administered in unit dosage form and that the unit dosage form would be adjusted accordingly by one skilled in the art to reflect the relative level of activity. The decision as to the particular dosage to be employed (and the number of times to be administered per day) is within the discretion of the physician, and may be varied by titration of the dosage to the particular circumstances of this invention to produce the desired therapeutic effect.

A suitable dose of the compound of Formula (Ia) or pharmaceutical composition thereof for a warm-blooded animal including humans, suffering from, or likely to suffer from any condition as described herein is an amount of active ingredient from about 0.01 mg/day to 2000 mg/day. For parenteral administration, the dose may be in the range of from about 0.1 mg/day to 500 mg/day, preferably from 1 mg/day to 250 mg/day for intravenous administration. For oral administration, the dose may be in the range of from about 0.1 mg/day to 2000 mg/day, preferably from about 4 mg/day to 200 mg/day, more preferably from about 2 to about 10 mg/day. The active ingredient will preferably be administered either continuously or in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances, including the condition to be treated, the chosen route of administration, the age, weight, and response of the individual patient, and the severity of the patient's symptoms.

The following examples are illustrative of the invention.

### EXAMPLE 1

### Preparation of {4-[2-(5-Methyl-2-phenyl-oxazol-4-yl)-ethoxy]-benzylamino}-acetic acid methyl ester hydrochloride

Option 1 for Addition of Starting Materials—One Pot Process.

Sequentially 100 mL of ethyl acetate, 8.3 g of triethylamine and 100 mL of methanol were added to the combined solids, 20 g of 4-[2-(5-methyl-2-phenyl-oxazol-4-yl)-ethoxy]-benzaldehyde and 10.1 g of glycine methyl ester hydrochloride in a 250 mL Buchi hydrogenation reactor under inert atmosphere

Option 2 for Addition of Starting Materials.

8.3 g of triethylamine was added to the solution of 10.1 g of glycine methyl ester hydrochloride in 100 mL methanol under inert atmosphere. The mixture was stirred at 20-25° C. until dissolution of glycine methyl ester hydrochloride was complete. This free glycine methyl ester solution was transferred to a 250 mL Buchi hydrogenation reactor containing the slurry solution of 20 g of 4-[2-(5-methyl-2-phenyl-oxazol-4-yl)-ethoxy]-benzaldehyde in 100 mL of ethyl acetate under inert atmosphere.

After the reaction mixture was stirred for 2 hours at 20-25° C. to form the intermediary Schiff base, the catalyst, 0.6 g of 5% Pd/C was added under inert conditions and hydrogen gas was introduced in the reactor. The reaction mixture was stirred for 30 min at 35-45° C. under 30-45 psi of hydrogen gas. Upon completion, the catalyst was removed by filtration under inert conditions. The filtrate was diluted with 200 mL˜16 w/w % aqueous sodium chloride solution and the lower aqueous layer was extracted with 100 mL ethyl acetate. The combined organic phase was washed with 200 mL˜16 w/w % aqueous sodium chloride solution. If the residual palladium content of the rich organic phase was determined to be >25 ppm, an optional filtration of the organic phase through carbon impregnated Zeta pads was used to reduce the residual palladium. The rich organic solution was distilled to remove trace water below 1.2 w/w % in KF and methanol. Ethyl acetate was added to the rich organic solution up to 300 mL followed by addition of 12.5 g methanol. The solution was heated to 38-45° C. and 8.26 mL of chlorotrimethylsilane was added at 38-50° C. following cubic addition profile for maximizing removal of organic impurities and for optimal particle size control that facilitate filtration. The crystal slurry was held at 35-50° C. for 30 min and cooled to 20-25° C. over an hour. After a two hour hold at 20-25° C., the product was filtered and washed twice with ethyl acetate (60 mL each). The product was dried to an LOD<2% in a vacuum oven below 50° C. 24.3 g (90 M %) of {4-[2-(5-methyl-2-phenyl-oxazol-4-yl)-ethoxy]-benzylamino}-acetic acid methyl ester hydrochloride was obtained as a white to off-white crystalline solid with HPLC AP of 99.4.

### EXAMPLE 2

### Optional Recrystallization of the Product Obtained in Example 1

Recrystallization was carried out to improve product quality and/or remove particulate matter. 28.5 g of {4-[2-(5-Methyl-2-phenyl-oxazol-4-yl)-ethoxy]-benzylamino}-acetic acid methyl ester hydrochloride (HPLC AP 97.8%) was mixed with 34.1 mL of methanol. The slurry solution was warmed to 45-58° C. to achieve complete dissolution. The product rich solution was polish filtered with a Buchner funnel equipped with Whatman #1 filter paper. During the polish filtration step the reactor temperature was maintained at 45-58° C. to prevent crystallization. 39 mL of ethyl acetate was added to the product rich solution at 55° C. The solution was cooled to 40-45° C. over 15 min and stirred for 30 min at 40-45° C. At 40-43° C., the solution gets cloudy. 354 mL of ethyl acetate was added following cubic addition profile for 2.5 hours at 40-45° C., Once addition of ethyl acetate was complete, the slurry was cooled to 20-25° C. and stirred for 2 hours at 20-25° C. The slurry was filtered and the product filter cake was washed with two time ethyl acetate (85 mL each). The product was dried under vacuum below 50° C. {4-[2-(5-Methyl-2-phenyl-oxazol-4-yl)-ethoxy]-benzylamino}-acetic acid methyl ester hydrochloride was isolated as a white to off-white crystalline solid (24.6 g; 86.3 M % yield “as is”; HPLC AP of 99.5)

### EXAMPLE 3

### Preparation of N-[(4-Methoxyphenoxy)carbonyl]-N-[[4-[2-(5-methyl-2-phenyl-4-oxazolyl)ethoxy]phenyl]methyl]glycine

15 g of {4-[2-(5-methyl-2-phenyl-oxazol-4-yl)-ethoxy]-benzylamino}-acetic acid methyl ester hydrochloride (36.1 mmol), 15 g of dibasic potassium phosphate (K₂HPO₄) in 150 mL of water, and 50 mL of tetrahydrofuran were mixed together at 20° C. to 30° C. 6 mL of 4-methoxyphenyl chloroformate was added to the reaction mixture to produce N-[(4-methoxyphenoxy)carbonyl]-N-[[4-[2-(5-methyl-2-phenyl-4-oxazolyl)-ethoxy]phenyl]methyl]glycine methyl ester. 30 mL of 10 N NaOH was added to the reaction mixture at 40° C. to 50° C. to initiate hydrolysis of N-[(4-methoxyphenoxy)carbonyl]-N-[[4-[2-(5-methyl-2-phenyl-4-oxazolyl)-ethoxy]phenyl]methyl]glycine methyl ester. The reaction mixture was heated to about 40° C. to 45° C. and stirred for about 2 hours, and thereafter neutralized to a pH of about 6.5 to 7.5 with the addition of 21 mL ca. 6M phosphoric acid. The reaction mixture was diluted with ethanol and processed through phase separation. The organic layer obtained from the phase separation was diluted with 22.5 mL of water and the resulting mixture was acidified with 12 mL 6M phosphoric acid to produce a pH of less than 3.5 at about 40° C. to 50° C. The reaction mixture in the form of a slurry was stirred for about 1 hour and diluted with 187.5 mL of water. The reaction mixture was stirred for an additional hour at about 40° C. to 50° C. Thereafter, the reaction mixture was cooled to about 18° C. to 23° C. and filtered to isolate the product. The isolated product was washed with 60 mL of 3:1 ethanol to water solution, followed by 60 mL of water (three times) and dried at less than 80° C. under reduced pressure to yield N-[[4-methoxyphenoxy)carbonyl]-N-[(4-[2-(5-methyl-2-phenyl-4-oxazolyl)-ethoxy]phenyl]methyl]glycine for 90% to 95% overall yield.

### EXAMPLE 4

### Alternative Process for Synthesizing N-[(4-Methoxyphenoxy)carbonyl]-N-[[4-[2-(5-methyl-2-phenyl-4-oxazolyl)-ethoxy]phenyl]methyl]glycine

50 g of {4-[2-(5-Methyl-2-phenyl-oxazol-4-yl)-ethoxy]-benzylamino}-acetic acid methyl ester hydrochloride was mixed with 100 mL of tetrahydrofuran and aqueous K₂HPO₄ (50 g dissolved in 200 mL of water). The resulting slurry was warmed to 30° C. to 40° C. 20 mL of 4-methoxychloroformate was added to the reaction mixture over 5 to 15 minutes while the temperature was maintained at about 30° C. to 40° C. The reaction mixture was stirred and monitored by HPLC analysis to ensure completion of the reaction. The reaction was completed within 5 minutes of stir time.

10M aqueous NaOH (100 mL) was added to the reaction mixture while maintaining the temperature of about 30° C. to 50° C. The reaction mixture was stirred as the reaction was monitored for completion. The reaction temperature was observed to rise to about 40° C. to 45° C. upon the addition of 10M aqueous NaOH. The reaction completion was indicated by HPLC analysis. The reaction was complete within 2 to 3 hours of stir time.

A dilute solution of H₃PO₄ (70 mL solution containing 85 weight % H₃PO₄ (27 mL) and q.s. with water to 70 mL) was added to the reaction mixture followed by the addition of 200 mL of ethanol (190 proof). The reaction mixture was stirred while the temperature was maintained at about 40° C. to 45° C. to yield a clear light orange bi-phasic mixture. Agitation was stopped and an organic phase was separated. The organic phase was then polish filtered followed by the sequential addition of 300 mL of ethanol (190 proof), diluted with 50-100 ml water. The pH of the mixture was adjusted to <3.5 with 40 mL of dilute H₃PO₄ (150 mL solution containing 85 weight % H₃PO₄ (58 mL and q.s. with water to 150 mL)). The resulting slurry was stirred for about 1 hour at a temperature of from about 40° C. to 50° C. Crystallization commenced within 10 minutes of stir time. The pH of the reaction mixture remained at <3.5.

If crystallization does not commence, the slurry may be seeded with Form N1 crystals of muraglitazar.

600-650 mL of water was added to the slurry and stirred for about 1 hour. The slurry was cooled to about 18° C. to 23° C. over a 1-hour period. The product slurry was filtered over a 11 cm Buchner filter equipped with Whatman #1 filter paper. Approximately 2 L of slurry was filtered in about 7 minutes. The product filter cake was successively washed with ethanol (190 proof):water (200 mL, 3:1 v/v) and three times with water (200 mL each). The product was dried under vacuum at a temperature≦80° C. to a final KF<0.3 wt %. N-[(4-Methoxyphenoxy)carbonyl]-N-[[4-[2-(5-methyl-2-phenyl-4-oxazolyl)-ethoxy]phenyl]methyl]glycine was isolated as an off-white powder (59.0 g; 95.0 M % yield “as is”, HPLC AP 99.7+, corrected purity of 99+).

### EXAMPLE 5

### Optional Product Slurry Sonication Technique

An in-process sonication technique was used to eliminate the need for milling the isolated/dried product of Example 3. Prior to the filtration described in Example 3, the product slurry was passed through a flow cell containing a 12-inch radial sonic resonator (Telsonic Sonoreactor 20 kHz) at a flow rate of about 2 to 4 liters per minute under pressure of 25 psig or greater with an average power input of at least 700 watts. Dry powder laser light scattering analysis (Malvern Mastersizer 2000) of the resulting material indicated a D(v, 0.9) of less than 20 μm for the 90^{th} percentile of particles and a D[4,3] of less than 23 μm for the volume weighted mean.

### EXAMPLE 6

### Optional Recrystallization of the Product Obtained in Example 3

Recrystallization was carried out to improve product quality and/or remove particulate matter. 15 g of N-[(4-methoxyphenoxy)carbonyl]-N-[[4-[2-(5-methyl-2-phenyl-4-oxazolyl)ethoxy]phenylmethyl]glycine was mixed with 135 mL of ethanol (190 proof) to yield a slurry. The slurry was heated to a temperature of about 75° C. to 80° C. to achieve complete dissolution. The product rich solution was polish filtered with a Buchner funnel equipped with Whatman #1 filter paper. During the polish filtration step the temperature was maintained above 50° C. to prevent crystallization. The filter funnel was washed with 33 mL of ethanol (190 proof) and combined with the filtrate. The product rich solution was cooled to about 40° C. to 45° C. Seed crystals of N-[(4-methoxyphenoxy)carbonyl]-N-[[4-[2-(5-methyl-2-phenyl-4-oxazolyl)ethoxy]phenyl]methyl]glycine (0.01 to 0.02 g) were added to the solution. The slurry was stirred for about one hour. The slurry was cooled to 18° C. to 23° C. over at least one hour. The slurry was then filtered and the product filter cake was successively washed with ethanol (190 proof):water (200 mL, 3:1) and three times with water (200 mL each). The product was dried under vacuum at a temperature≦80° C. N-[(4-methoxyphenoxy)carbonyl]-N-[[4-[2-(5-methyl-2-phenyl-4-oxazolyl)-ethoxy]phenyl]methyl]glycine was isolated as an off-white powder (12.8 g; 85.5 M % yield “as is”; HPLC AP of 99.8%, corrected purity of 99.7+).

### EXAMPLE 7

### Alternative preparation of N-[(4-Methoxyphenoxy)carbonyl]-N-[[4-[2-(5-methyl-2-phenyl-4-oxazolyl)ethoxy]phenyl]methyl]glycine

15 g of {4-[2-(5-methyl-2-phenyl-oxazol-4-yl)-ethoxy]-benzylamino}-acetic acid methyl ester hydrochloride (36.1 mmol), 15 g of dibasic potassium phosphate (K₂HPO₄) in 150 mL of water, and 50 mL of tetrahydrofuran were mixed together at 20° C. to 30° C. 6 mL of 4-methoxyphenyl chloroformate was added to the reaction mixture over 3 minutes to produce N-[(4-methoxyphenoxy)carbonyl]-N-[[4-[2-(5-methyl-2-phenyl-4-oxazolyl)ethoxy]phenyl]methyl]glycine methyl ester. The reaction mixture was stirred and monitored by HPLC analysis to ensure completion of the reaction. The reaction was completed within 30 minutes of stir time. 30 mL of 10N NaOH solution was added to the reaction mixture and the mixture heated to about 40° C. to 45° C. The reaction mixture was monitored by HPLC analysis to ensure completion of the reaction. The reaction was completed within 2 to 3 hours after addition of the 10N NaOH solution.

The reaction mixture was cooled to 20° C. to 25° C. and 24 mL of 6N HCl solution was added to the mixture and pH of the mixture was checked. Additional 6N HCl (6 mL) was added to bring the pH of the reaction mixture to about 6.5 to 7.5. Ethyl acetate (80 mL) was added to the vessel followed by 30 mL of 6N HCl. The pH of a sample taken from the aqueous layer was 1.3. Stirring was halted to allow the upper organic layer and the lower aqueous layers to separate. The aqueous layer was drained from the vessel. Water (80 mL) was added to the vessel and mixed with the organic layer. Stirring was halted to effect a phase split, and the lower aqueous layer was removed from the reactor. This procedure was repeated one additional time. The remaining organic solution was filtered through a 42.5 mm Whatman #1 filter paper in a Büchner funnel and the vessel and filter were rinsed with 80 mL of ethyl acetate.

The combined organic solutions were transferred to a jacketed chemical reactor for distillation. The jacketed chemical reactor had been previously calibrated with water to measure the internal volume on the reactor jacket. The volume of organic solution for the distillation was measured at 210 mL. The water content of the organic solution was measured via Karl Fischer (KF) titration. The KF of the solution was 3.9%. The solution was distilled under vacuum (400-500 torr, 45° C. to 55° C.). Ethyl acetate was periodically added to maintain the reactor volume between 90 mL and 190 mL and the KF of the solution was monitored by taking samples. Distillation was stopped when the KF of the solution was below 0.1%. The reactor volume was 110 mL. During the distillation, some crystals were evident on the sides of the reactor. The ethyl acetate solution was heated to reflux (78° C.) to dissolve the crystals. The ethyl acetate solution was cooled to 75° C. and then n-heptane was added slowly, keeping the reaction vessel temperature between 65° C. and 75° C. Addition of n-heptane was stopped when the cloudiness of the solution persisted for more than five minutes. Upon addition of the 60 mL of n-heptane, clouding of the solution was evident by visual inspection. A circulating bath was programmed to hold the reactor jacket at 75° C. for one hour, then cool to 20° C. over five hours. Upon cooling, a thick slurry was evident. The slurry was filtered over a 70 mm Whatman #1 filter paper in a Büchner funnel. The resulting cake was washed twice with a 1:1 (v/v) mixture of ethyl acetate:n-heptane (50 mL per wash). The cake was left to air dry or alternatively, may be dried in a vacuum oven (50 to 150 torr, 20° C. to 40° C.). 16.3 g (87.3% yield) of an off-white solid was isolated. Analysis by HPLC indicated a 99.6 area percent (AP).

To remove individual impurities and ensure the correct crystalline form, 150 mL of SDA3A ethanol was added to 10 g of the above solid and the mixture heated to 60° C. to 65° C. to effect dissolution. The solution was cooled to 50° C. and filtered through a 42.5 mm Whatman #1 filter paper in a Büchner funnel and the vessel and filter were rinsed with 30 mL of SDA3A ethanol. The solution was distilled under reduced pressure in a temperature range of 40° C. to 50° C., until the solution volume was reduced to 90 mL wherein precipitation was evident.

The resulting slurry was heated to 60° C. to 65° C. and optionally, methanol (5 mL) can be added to aid in refluxing and washing solids off of the side of the vessel. The resulting slurry was held at 60° C. to 65° C. for 30 minutes and then cooled to 20° C. to 25° C. over 3 hours. The slurry was cooled to 0° C. and filtered over a 55 mm Whatman #1 filter paper in a Büchner funnel. The resulting cake was washed with 40 mL of cold (0° C. to 10° C.) SDA3A ethanol. The cake was dried in a vacuum oven (50 to 150 torr, 20° C. to 40° C.). 9.3 g (92.3% recovery) of a white solid was isolated. Analysis by HPLC indicated a 99.8 area percent (AP), with no individual impurities above 0.1%.

### Polymorphic Form

The present invention provides a free-acid polymorphic form of compound Ia, herein referred to as the N-1 form, that has been isolated and/or identified.

The ability of a compound to exist in different crystal structures is known as polymorphism. As used herein “polymorph” refers to crystalline forms having the same chemical composition but different spatial arrangements of the molecules, atoms, and/or ions forming the crystal. While polymorphs have the same chemical composition, they differ in packing and geometrical arrangement, and may exhibit different physical properties such as melting point, shape, color, density, hardness, deformability, stability, dissolution, and the like. Depending on their temperature-stability relationship, two polymorphs may be either monotropic or enantiotropic. For a monotropic system, the relative stability between the two solid phases remains unchanged as the temperature is changed. In contrast, in an enantiotropic system there exists a transition temperature at which the stability of the two phases reverse. (Theory and Origin of Polymorphism in “Polymorphism in Pharmaceutical Solids” (1999) ISBN: )-8247-0237).

Samples of the crystalline forms may be provided with substantially pure phase homogeneity, indicating the presence of a dominant amount of a single crystalline form and optionally minor amounts of one or more other crystalline forms. The presence of more than one crystalline form in a sample may be determined by techniques such as powder X-ray diffraction (PXRD) or solid state nuclear magnetic resonance spectroscopy (SSNMR). For example, the presence of extra peaks in the comparison of an experimentally measured PXRD pattern (observed) with a simulated PXRD pattern (calculated) may indicate more than one crystalline form in the sample. The simulated PXRD may be calculated from single crystal X-ray data. (see Smith, D. K., “A FORTRAN Program for Calculating X-Ray Powder Diffraction Patterns,” Lawrence Radiation Laboratory, Livermore, Calif., UCRL-7196, April 1963; see also Yin. S.; Scaringe, R. P.; DiMarco, J.; Galella, M. and Gougoutas, J. Z., American Pharmaceutical Review, 2003, 6, 2, 80). Preferably, the crystalline form has substantially pure phase homogeneity as indicated by less than 10%, preferably less than 5%, and more preferably less than 2% of the total peak area in the experimentally measured PXRD pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

The various forms described herein may be distinguishable from one another through the use of various analytical techniques known to one of ordinary skill in the art. Such techniques include, but are not limited to, solid state nuclear magnetic resonance (SSNMR) spectroscopy, X-ray powder diffraction (PXRD), differential scanning calorimetry (DSC), and/or thermogravimetric analysis (TGA).

### Preparation of Polymorphic Form

Procedures for the preparation of crystalline forms are known in the art. The crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (counter solvents) to the solvent mixture. High throughput crystallization techniques may be employed to prepare crystalline forms including polymorphs.

Crystals of drugs, including polymorphs, methods of preparation, and characterization of drug crystals are discussed in Solid-State Chemistry of Drugs, S. R. Byrn, R. R. Pfeiffer, and J. G. Stowell, 2^{nd} Edition, SSCI, West Lafayette, Ind., 1999.

Seed crystals may be added to any crystallization mixture to promote crystallization. As will be clear to the skilled artisan, seeding is used as a means of controlling growth of a particular crystalline form or as a means of controlling the particle size distribution of the crystalline product. Accordingly, calculation of the amount of seeds needed depends on the size of the seed available and the desired size of an average product particle as described, for example, in “Programmed cooling of batch crystallizers,” J. W. Mullin and J. Nyvlt, Chemical Engineering Science, 1971, 26, 369-377. In general, seeds of small size are needed to effectively control the growth of crystals in the batch. Seeds of small size may be generated by sieving, milling, or micronizing of larger crystals, or by micro-crystallization of solutions. Care should be taken that milling or micronizing of crystals does not result in any change in crystallinity from the desired crystal form (i.e. change to amorphous or to another polymorph).

As used herein, the term “room temperature” or “RT” denotes an ambient temperature from 20 to 25° C. (68-77° F.).

In general, the preparation of compounds of formula Ia is described in Example 230 of U.S. Pat. No. 6,414,002, which is herein incorporated by reference in its entirety.

Preparation of the N-1 polymorph of N-[(4-methoxyphenoxy)carbonyl]-N-[[4-[2-(5-methyl-2-phenyl-4-oxazolyl)ethoxy]phenyl]methyl]glycine may be completed as described in Example 4 above.

### Polymorphic Characterization

Polymorphic forms equivalent to the polymorphic forms described below and claimed herein may demonstrate similar, yet non-identical, analytical characteristics within a reasonable range of error, depending on test conditions, purity, equipment and other common variables known to those skilled in the art.

Accordingly, it will be apparent to those skilled in the art that various modifications and variations can be made in the present invention without departing from the scope and sprit of the invention. Other embodiments of the invention will be apparent to those skilled in the art from consideration of the specification and practice of the invention disclosed herein. Applicants intend that the specification and examples be considered as exemplary, but not limiting in scope.

X-Ray Powder Diffraction

One of ordinary skill in the art will appreciate that a powder X-ray diffraction pattern may be obtained with a measurement error that is dependent upon the measurement conditions employed. In particular, it is generally known that intensities in a X-ray powder diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional powder X-ray powder diffraction pattern is typically about 5% or less, and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray powder diffraction patterns depicted in the accompanying Figures disclosed herein. Any crystal forms that provide powder X-ray diffraction patterns substantially identical to those disclosed in the accompanying Figures fall within the scope of the present invention. The ability to ascertain substantial identities of X-ray powder diffraction patterns is within the purview of one of ordinary skill in the art.

About 200 mg were packed into a Philips powder X-ray diffraction (PXRD) sample holder. The sample was tranferred to a Philips MPD unit (45 KV, 40 mA, Cu Kα). Data were collected at room temperature in the 2 to 32 2-θ (2-theta) range (continuous scanning mode, scanning rate 0.03 degrees/sec., auto divergence and anti scatter slits, receiving slit: 0.2 mm, sample spinner: ON)

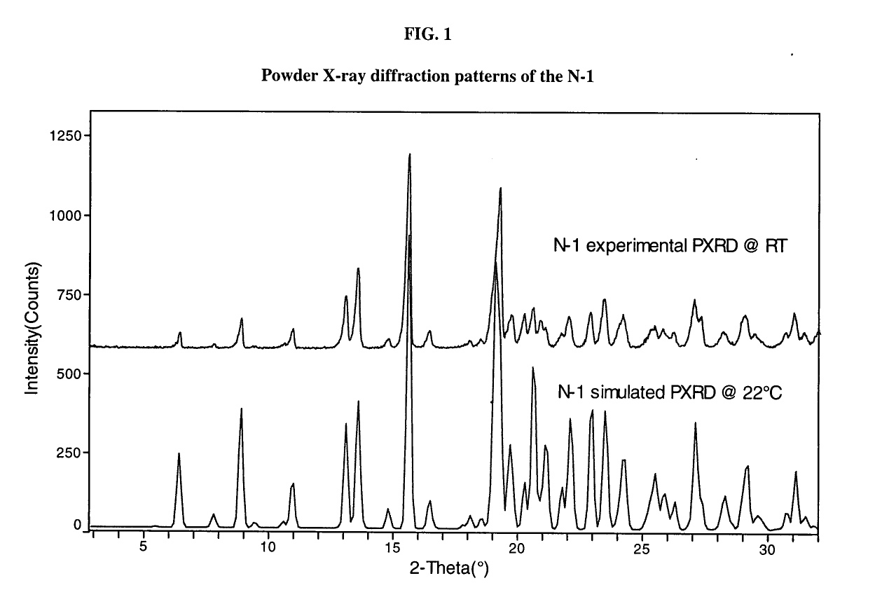

The powder X-ray diffraction pattern for the N-1 form is illustrated in FIG. 1. Selected diffraction peak positions (degrees 2θ±0.2) for N-1 are shown in Table 1 below. Characteristic diffraction peak positions (degrees 2θ±0.1)@ RT, based on a high quality pattern collected with a diffractometer (CuKα) with a spinning capillary with 2θ calibrated with a National Institute of Standards and Technology methodology, other suitable standard known to those skilled in the art. The relative intensities, however, may change depending on the crystal size and morphology.

TABLE 1
Selected PXRD Peaks (2θ, °)
N-1

6.4
8.9
11.0
13.1
13.6
15.6
19.1
20.6
22.1
23.0

Solid-State Nuclear Magnetic Resonance

The N-1 form was characterized by solid state NMR techniques.

All solid-state C-13 NMR measurements were made with a Bruker DSX-400, 400 MHz NMR spectrometer. High resolution spectra were obtained using high-power proton decoupling and the TPPM pulse sequence and ramp amplitude cross-polarization (RAMP-CP) with magic-angle spinning (MAS) at approximately 15 kHz (N-1 spectrum) (A. E. Bennett et al, J. Chem. Phys., 1995, 103, 6951),(G. Metz, X. Wu and S. O. Smith, J. Magn. Reson. A, 1994, 110, 219-227). Approximately 70 mg of sample, packed into a canister-design zirconia rotor was used for each experiment. Chemical shifts (δ) were referenced to external adamantane with the high frequency resonance being set to 38.56 ppm, relative to tetramethylsilane (TMS) at zero. (W. L. Earl and D. L. VanderHart, J. Magn. Reson., 1982, 48, 35-54).

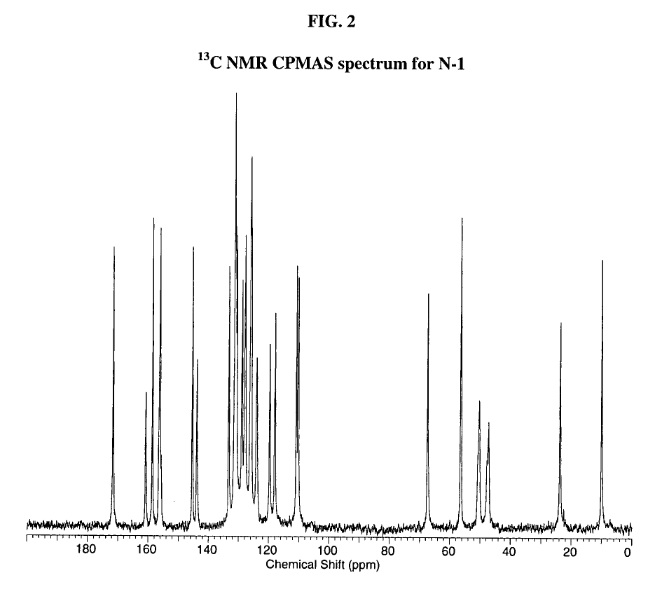

The resulting ¹³C NMR CPMAS spectrum for the N-1 form is shown in FIG. 2.

The major resonance peaks for the solid state carbon spectrum of form N-1 are listed below in Table 2. Crystal forms demonstrating substantially similar ¹³C NMR peak positions, wherein “substantially similar” means 10 to 15% of dimensionless value, are deemed to fall within the scope of the invention (i.e., equivalent to the N-1 form peaks illustrated below).

TABLE 2
SSNMR peak positions/δ (in ppm) relative to TMS
N-1

10.1
23.8
47.4
50.4
56.5
67.4
110.3, 110.9*
118.0, 119.8*
124.1
126.1
128.0
129.0
130.8
131.1
131.4
133.3
144.0
145.5
155.8
156.3
158.6
160.9
171.7
*These peaks are doubled due to quadrupolar coupling to ¹⁴N nuclei. This may vary significantly, depending upon the instrument magnetic field. These data are strictly valid for a 400 MHz spectrophotometer.
Duplicates without an “*” illustrated for N-3 indicate two molecules in the same asymmetric unit.

Duplicates without an “*” illustrated for N-3 indicate two molecules in the same asymmetric unit.

Thermal Gravimetric Analysis

Thermal gravimetric analysis (TGA) experiments were performed in a TA Instruments™ model Q500 or 2950. The sample (about 10-30 mg) was placed in a platinum pan previously tared. The weight of the sample was measured accurately and recorded to a thousand of a milligram by the instrument The furnace was purged with nitrogen gas at 100 mL/min. Data were collected between room temperature and 300° C. at 10° C./min heating rate.

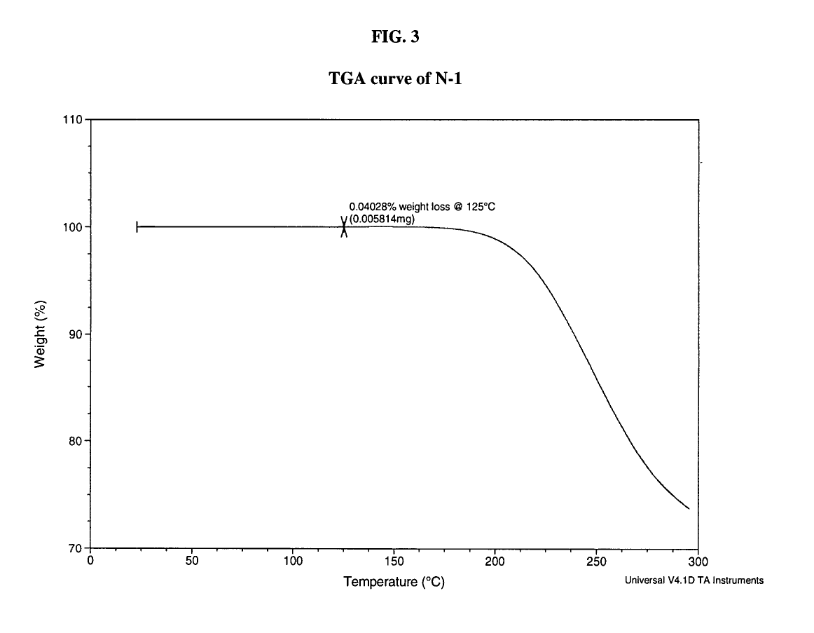

A TGA curve for the N-1 form is shown in FIG. 3.

Differential Scanning Calorimetry

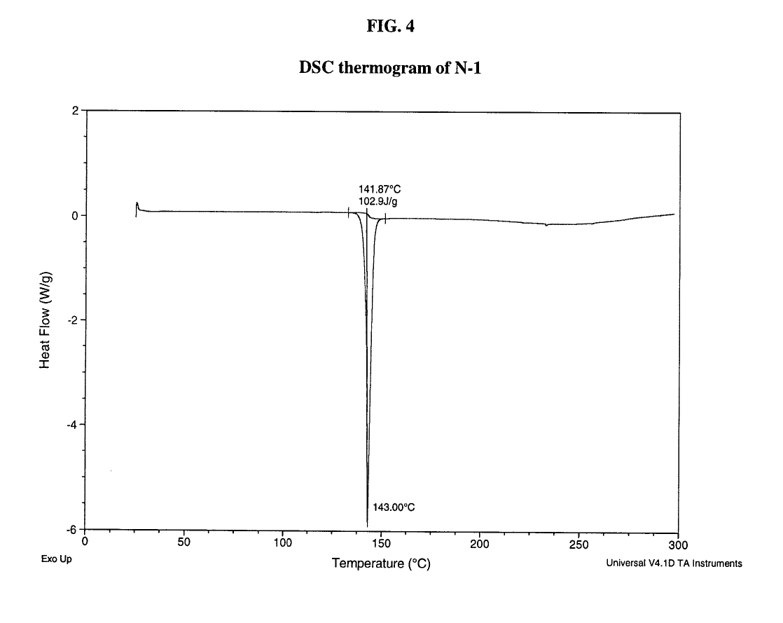

The solid state thermal behavior of the N-1 form was investigated by differential scanning calorimetry (DSC). The DSC curve for the N-1 form is shown in FIG. 4.

DSC (open pan) experiments were performed in a TA Instruments™ model Q1000 or 2920. The sample (about 2-6 mg) was weighed in an aluminum pan and recorded accurately recorded to a hundredth of a milligram, and transferred to the DSC. The instrument was purged with nitrogen gas at 50 mL/min. Data were collected between room temperature and 300° C. at 10° C./min heating rate. The plot was made with the endothermic peaks pointing down.

One of skill in the art will however, note that in DSC measurement there is a certain degree of variability in actual measured onset and peak temperatures, depending on rate of heating, crystal shape and purity, and other measurement parameters.

Moisture Sorption Isotherms

Moisture sorption isotherms were collected in a VTI SGA-100 Symmetric Vapor Analyzer using approximately 10 mg of sample. The sample was dried at 60° C. until the loss rate of 0.0005 wt %/min was obtained for 10 minutes. The sample was tested at 25° C. and 3 or 4, 5, 15, 25, 35, 45, 50, 65, 75, 85, and 95% relative humidity (RH). Equilibration at each RH was reached when the rate of 0.0003 wt %/min for 35 minutes was achieved or a maximum of 600 minutes.

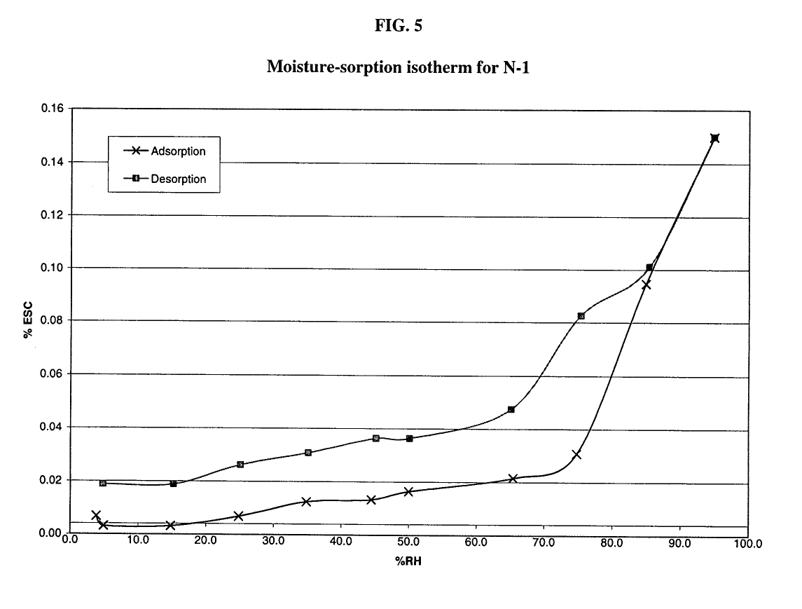

Moisture sorption isotherms for the N-1 form is shown in FIG. 5.

Single Crystal X-Ray Analysis

A single crystal for the N-1 form was obtained and investigated by x-ray diffraction.

Data were collected on a Bruker-Nonius¹ CAD4 serial diffractometer. Unit cell parameters were obtained through least-squares analysis of the experimental diffractometer settings of 25 high-angle reflections. A detailed account of unit cells can be found in Chapter 3 of Stout & Jensen, “X-Ray Structure Determination: A Practical Guide”, (MacMillian, 1968). Intensities were measured using Cu Kα radiation (λ=1.5418 Å) at a constant temperature with the θ-2θ variable scan technique and were corrected only for Lorentz-polarization factors. Background counts were collected at the extremes of the scan for half of the time of the scan. Alternately, single crystal data were collected on a Bruker-Nonius Kappa CCD 2000 system using Cu Kα radiation (λ=1.5418 Å). Indexing and processing of the measured intensity data were carried out with the HKL2000 software package² in the Collect program suite.³
¹BRUKER AXS, Inc., 5465 East Cheryl Parkway Madison, Wis. 53711 USA.

²Otwinowski, Z. & Minor, W. (1997) in Macromolecular Crystallography, eds. Carter, W. C. Jr & Sweet, R. M. (Academic, N.Y.), Vol. 276, pp. 307-326.

³Collect Data collection and processing user interface: Collect: Data collection software, R. Hooft, Nonius B. V., 1998.

When indicated, crystals were cooled in the cold stream of an Oxford cryo system⁴ during data collection.
⁴Oxford Cryosystems Cryostream cooler: J. Cosier and A. M. Glazer, J. Appl. Cryst., 1986, 19, 105.

The structures were solved by direct methods and refined on the basis of observed reflections using either the SDP⁵software package with minor local modifications or the crystallographic package, MAXUS.⁶
⁵SDP, Structure Determination Package, Enraf-Nonius, Bohemia N.Y. 11716. Scattering factors, including ƒ′ and ƒ″, in the SDP software were taken from the “International Tables for Crystallography”, Kynoch Press, Birmingham, England, 1974; Vol. IV, Tables 2.2A and 2.3.1.

⁶maXus solution and refinement software suite: S. Mackay, C. J. Gilmore, C. Edwards, M. Tremayne, N. Stewart, K. Shankland. maXus: a computer program for the solution and refinement of crystal structures from diffraction data.

The derived atomic parameters (coordinates and temperature factors) were refined through full matrix least-squares. The function minimized in the refinements was Σ_{w}(|Fₒ|−|F_{c}|)² R is defined as Σ ∥Fₒ|−|F_{c}∥/Σ |Fₒ| while R_{w}=[Σ_{w}(|Fₒ|−|F_{c}|)²/Σ_{w}|Fₒ|²]^{1/2} where w is an appropriate weighting function based on errors in the observed intensities. Difference maps were examined at all stages of refinement. Hydrogens were introduced in idealized positions with isotropic temperature factors, but no hydrogen parameters were varied. Pertinent crystal, data collection and refinement are summarized in Table 3, below.

TABLE 3
Crystallographic Data

| Form | T | a(Å) | b(Å) | c(Å) | α° | β° | γ° | V(Å³) | Z′ | sg | dcalc | R |
|---|---|---|---|---|---|---|---|---|---|---|---|---|
| N-1 | 22 | 4.793(1) | 19.914(4) | 27.696(4) | 90.0 | 94.52(1) | 90.0 | 2635(1) | 1 | P2₁/c | 1.302 | .05 |

T = temp(° C.) for the crystallographic data.
Z′ = number of drug molecules per asymmetric unit
V = volume and sg = space group.

Numerical values illustrated within brackets ( ) for Table 3 above and Table 4 below, denote estimated standard deviations in least significant figures.

Table 4 sets forth the positional parameters and their estimated standard deviations for the N-1 form.

TABLE 4
Positional Parameters for form N-1 at RT

| Atom | x | y | z | B(iso) |
|---|---|---|---|---|
| O1 | −0.0801(6) | 0.2959(1) | 0.35087(9) | 4.35(7) |
| O1A | −0.4604(6) | 0.2630(2) | 0.3055(1) | 5.23(8) |
| O11 | 0.3050(6) | 0.5594(1) | 0.1171(1) | 4.35(7) |
| O17 | 0.2049(6) | 0.6962(1) | −0.00734(9) | 4.71(7) |
| O26 | −0.1582(6) | 0.2103(1) | 0.2159(1) | 4.99(7) |
| O26A | −0.4615(7) | 0.2584(2) | 0.1589(1) | 5.51(8) |
| O33 | −0.2334(6) | −0.0347(2) | 0.1237(1) | 5.49(8) |
| N3 | −0.2658(7) | 0.3184(2) | 0.2228(1) | 3.79(8) |
| N15 | 0.2552(6) | 0.7380(2) | 0.0664(1) | 3.64(8) |
| C1 | −0.2346(8) | 0.2894(2) | 0.3094(1) | 3.52(9) |
| C2 | −0.0902(9) | 0.3184(2) | 0.2678(1) | 3.8(1) |
| C4 | −0.4106(9) | 0.3809(2) | 0.2078(2) | 4.1(1) |
| C5 | −0.2201(8) | 0.4291(2) | 0.1845(1) | 3.6(1) |
| C6 | −0.141(1) | 0.4178(2) | 0.1382(2) | 4.5(1) |
| C7 | 0.0331(9) | 0.4619(2) | 0.1168(1) | 4.4(1) |
| C8 | 0.1349(8) | 0.5185(2) | 0.1415(1) | 3.7(1) |
| C9 | 0.0602(9) | 0.5304(2) | 0.1877(2) | 4.2(1) |
| C10 | −0.1179(9) | 0.4857(2) | 0.2085(1) | 4.2(1) |
| C12 | 0.4171(9) | 0.6174(2) | 0.1418(2) | 4.1(1) |
| C13 | 0.5939(9) | 0.6552(2) | 0.1081(2) | 4.3(1) |
| C14 | 0.4248(8) | 0.6808(2) | 0.0643(2) | 3.7(1) |
| C16 | 0.1323(9) | 0.7448(2) | 0.0233(1) | 3.7(1) |
| C18 | 0.394(1) | 0.6557(2) | 0.0198(2) | 4.7(1) |
| C19 | 0.514(1) | 0.5986(3) | −0.0051(2) | 7.9(2) |
| C20 | −0.0646(8) | 0.7958(2) | 0.0046(1) | 3.6(1) |
| C21 | −0.2289(9) | 0.7856(2) | −0.0384(2) | 4.5(1) |
| C22 | −0.415(1) | 0.8342(3) | −0.0557(2) | 5.0(1) |
| C23 | −0.438(1) | 0.8935(3) | −0.0309(2) | 5.6(1) |
| C24 | −0.272(1) | 0.9044(2) | 0.0113(2) | 6.1(1) |
| C25 | −0.088(1) | 0.8553(2) | 0.0295(2) | 5.0(1) |
| C26 | −0.3095(9) | 0.2625(2) | 0.1957(2) | 4.3(1) |
| C27 | −0.1889(9) | 0.1492(2) | 0.1911(1) | 4.2(1) |
| C28 | −0.376(1) | 0.1029(2) | 0.2057(2) | 4.8(1) |
| C29 | −0.396(1) | 0.0400(2) | 0.1840(2) | 4.8(1) |
| C30 | −0.2270(9) | 0.0254(2) | 0.1477(1) | 3.9(1) |
| C31 | −0.0422(9) | 0.0726(2) | 0.1324(2) | 4.5(1) |
| C32 | −0.0200(9) | 0.1347(2) | 0.1549(2) | 4.7(1) |
| C33 | −0.435(1) | −0.0835(2) | 0.1350(2) | 5.9(1) |
| H11 | −0.213 | 0.276 | 0.382 | 5.4 |

Hot-Stage Microscopy

The N-1 form was characterized by hot-stage microscopy.

Data were collected on a Mettler FP 82 HT Hot Stage or FP 84HT TA Microscopic Cell mounted on a microscope, using 400× nominal magnification and various filters. The heating rate was controlled at 10° C./min for the temperature range, ambient to 150° C. The crystals were observed visually for evidence of phase transformation, changes in birefringence, opacity, and melting etc.

Melt onset for N-1 was about 137° C. and melting was complete at about 145° C.

### Utilities and Combinations

A. Utilities

The compounds of Formula I and Ia possesses binding affinity at both PPAR alpha and PPAR gamma receptors, and therefore may be used in the treatment of diseases or disorders associated with PPAR activity.

Accordingly, the compound of the present invention can be administered to mammals, preferably humans, for the treatment of a variety of conditions and disorders, including, but not limited to, treating or delaying the progression or onset of diabetes(including Type I and Type II, impaired glucose tolerance, insulin resistance, and diabetic complications, such as nephropathy, retinopathy, neuropathy and cataracts), hyperglycemia, hyperinsulinemia, hypercholesterolemia, elevated blood levels of free fatty acids or glycerol, hyperlipidemia, hypertriglyceridemia, obesity, wound healing, tissue ischemia, atherosclerosis and hypertension. The compound of the present invention may also be utilized to increase the blood levels of high density lipoprotein (HDL).

In addition, the conditions, diseases, and maladies collectively referenced to as “Syndrome X” or Metabolic Syndrome as detailed in Johannsson J. Clin. Endocrinol. Metab., 82, 727-34 (1997), may be treated employing the compound of the present invention.

B. Combinations

The present invention includes within its scope pharmaceutical compositions comprising, as an active ingredient, a therapeutically effective amount of a compound of formula Ia, alone or in combination with a pharmaceutical carrier or diluent. Optionally, the compound of the present invention can be utilized as an individual treatment, or utilized in combination with one or more other therapeutic agent(s).

Other “therapeutic agent(s)” suitable for combination with the compound of the present invention include, but are not limited to, known therapeutic agents useful in the treatment of the aforementioned disorders including: anti-diabetic agents; anti-hyperglycemic agents; hypolipidemic/lipid lowering agents; anti-obesity agents; anti-hypertensive agents and appetite suppressants.

Examples of the aforementioned therapeutic agents, and specific examples suitable for use in combination with the compounds disclosed herein, are described in U.S. Pat. No. 6,414,002, incorporated by reference herein.

Numerous modifications and variations of the present invention are possible in light of the above teachings. It is therefore to be understood that within the scope of the appended claims, the invention may be practiced otherwise than as specifically described herein.

1. A process of preparing the compound of Formula (I), or a pharmaceutically acceptable salt thereof, and isomers thereof, 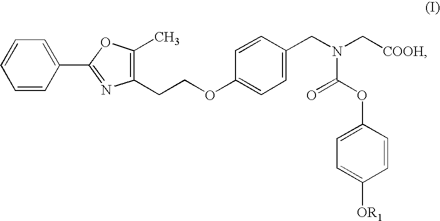
wherein R₁ is selected from alkyl, aryl, alkenyl, alkynyl, alkyloxy(halo)aryl, alkyl(halo)aryl or cycloalkylaryl, comprising:
reacting an acid salt of the compound of Formula (II) 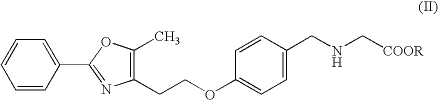
wherein R is alkyl, with the compound of Formula (III) 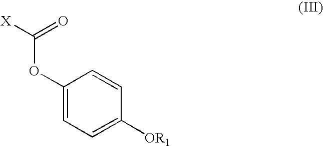
wherein X is selected from Cl, Br or I, and R₁ is as defined above, in the presence of a base, to yield the compound of Formula (IV) 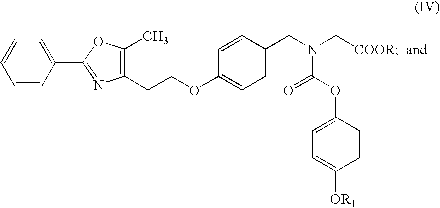
hydrolyzing the compound of Formula (IV) to yield the compound of Formula (I).

2. The process of claim 1 wherein the acid salt of the compound of Formula (II) is derived from a salt-forming reagent selected from the group consisting of an organic acid, an inorganic acid, alkylchlorosilane in the presence of an alcohol, and combinations thereof.

3. The process of claim 1 wherein R is alkyl or a prodrug ester, and R₁ is alkyl, and R is alkyl.

4. The process of claim 1 wherein X is Cl and R₁ is methyl.

5. The process of claim 1 wherein the reaction of the acid salt of the compound of Formula (II) with the compound of Formula (III), and the hydrolysis of the compound of formula (IV) are completed in the same reaction vessel.

6. The process of claim 1 wherein the compound of Formula (I) is recovered in solid form and is further subjected to a sonication process.

7. The process of claim 1 wherein the reaction of the acid salt of the compound of Formula (II) is reacted with the compound of Formula (III) using a buffered aqueous solvent system such as dibasic potassium phosphate to facilitate the formation of compound of Formula (IV) whilst minimizing the formation of reaction by-products.

8. The process of claim 1 further comprising crystallizing the compound of Formula (I) by the addition of an acid and/or alcohol, and water to the hydrolysis reaction mixture of the compound of formula (IV) therefrom.

9. The process of claim 8 further comprising crystallizing the compound of Formula (I) by adding aqueous acid and/or alcohol to the reaction mixture, separating out an organic phase, adding water and alcohol to the organic phase, adjusting pH of the organic phase to less than 3.5 by adding aqueous acid to the organic phase and causing the Formula (I) compound to crystallize out.

10. The process of claim 8 further comprising inducing crystallization of the compound of Formula (I) by the addition of a crystallizing agent.

11. The process of claim 1 further comprising crystallizing the compound of Formula (I) by adding acid to the reaction mixture to bring the pH to about 6.5 to about 7.5, separating out organic phase, adding solvent to the organic phase and heating the mixture to effect crystallization.

12. A process of preparing an acid salt of the compound of Formula (II) 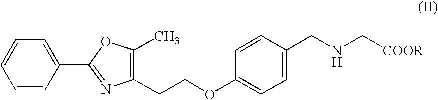
wherein R is alkyl comprising:
reacting the compound of Formula (V) 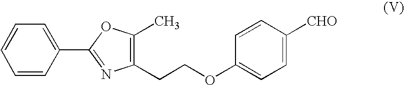
with a glycine ester acid salt to yield a Schiff base of Formula (VI) 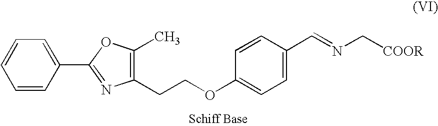
catalytically reducing the Schiff base of Formula (VI) to yield the compound of Formula (II) 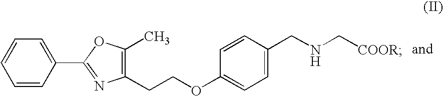
treating compound of Formula (II) with an acid salt-forming reagent to yield the acid salt of the compound of Formula (II).

13. The process of claim 12 comprising of conducting the reaction between the compound of Formula (V) and the glycine ester acid salt in the presence of a base which is a tertiary amine.

14. The process of claim 13 wherein the glycine ester acid salt is glycine methyl ester HCl.

15. The process of claim 12 wherein the formation of the Schiff base and the reduction of the Schiff base is done in the same reaction vessel.

16. The process of claim 12 wherein the reduction of the Schiff base is done with a metal hydride, selected from alkali metal boranes, a palladium metal catalyst supported on carbon (Pd/C), or a platinum metal catalyst supported on carbon (Pt/C).

17. The process of claim 12 wherein the salt-forming reagent selected from the group consisting of an organic acid, an inorganic acid, organohalosilane in combination with an alcohol, and combinations thereof.

18. A process of preparing the compound of Formula (I), or a pharmaceutically acceptable salt thereof, and isomers thereof, 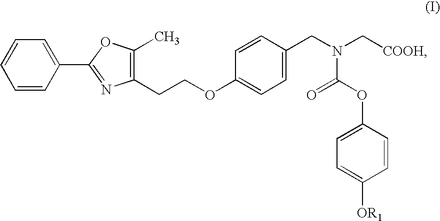
wherein R₁ is selected from alkyl, aryl, alkenyl, alkynyl, alkyloxy(halo)aryl, alkyl(halo)aryl or cycloalkylaryl, comprising:
reacting the compound of Formula (V) 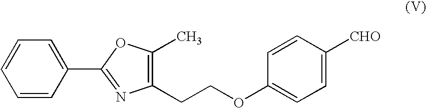
with a glycine ester acid salt to yield a Schiff base of Formula (VI) 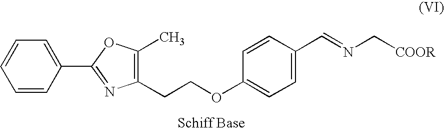
wherein R is alkyl,
catalytically reducing the Schiff base of Formula (VI) to yield the compound of Formula (II) 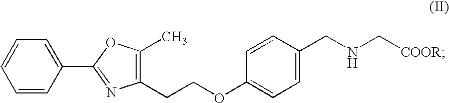
treating the compound of Formula (II) with an acid salt-forming reagent to yield the acid salt of the compound of Formula (II),
reacting the acid salt of the compound of Formula (II) with the compound of Formula (III) 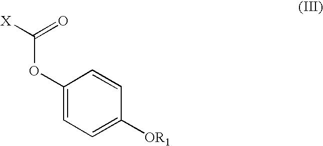
wherein X is selected from Cl, Br or I, and R₁ is as defined above, in the presence of a base, to yield the compound of Formula (IV) 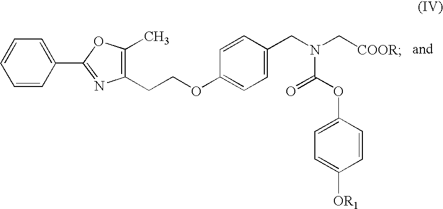
hydrolyzing the compound of Formula (IV) to yield the compound of Formula (I).

19. The process as defined in claim 18 wherein R₁ is CH₃, X is Cl and R is CH₃.

20. The process of claim 18 wherein preparation of the ester of compound of Formula (IV) and the subsequent reaction to provide the compound of Formula (I) are performed in the same reaction vessel.

21. A process of preparing the compound of Formula (Ia) 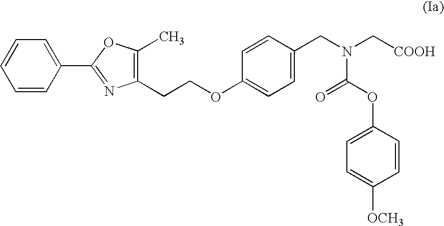
comprising:
reacting an acid salt of the compound of Formula (IIa) 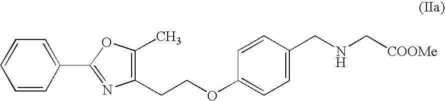
as defined in claim 24, with 4-methoxyphenyl chloroformate in the presence of a base to form the compound of Formula (IVa)
hydrolyzing the compound of Formula (IVa) 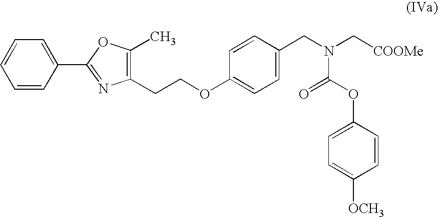
to yield a compound of Formula (Ia)

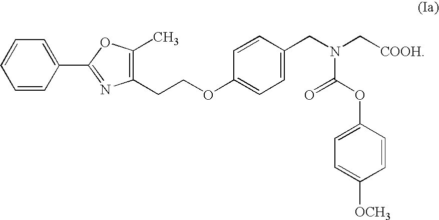

22. The process of claim 21 wherein the acid salt of the compound of Formula (II) is derived from a salt-forming reagent selected from the group consisting of an organic acid, an inorganic acid, organohalosilane in combination with an alcohol, and combinations thereof.

23. The process of claim 21 wherein the compound of Formula (IIa) is prepared by reacting the compound of Formula (V) 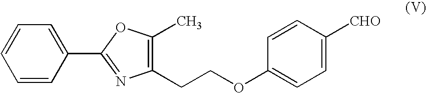
with glycine methyl ester acid salt to yield a Schiff base of Formula (VIa) 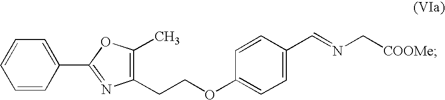
catalytically reducing the Schiff base of Formula (VIa) to yield a compound of Formula (IIa) 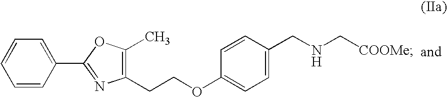
reacting the compound of Formula (IIa) with an acid salt-forming reagent to yield an acid salt of the compound of Formula (IIa).

24. A compound having the formula (IIa) 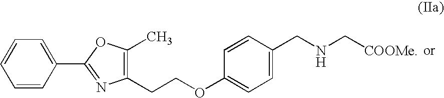
a compound having the formula (VIa) 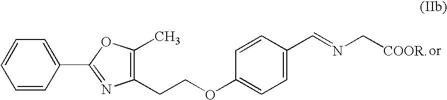
a compound having the formula (IVa)

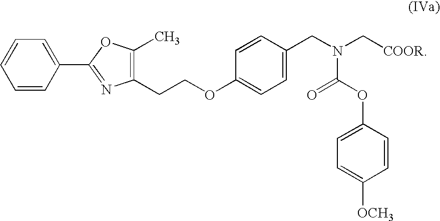

25. A crystalline form of

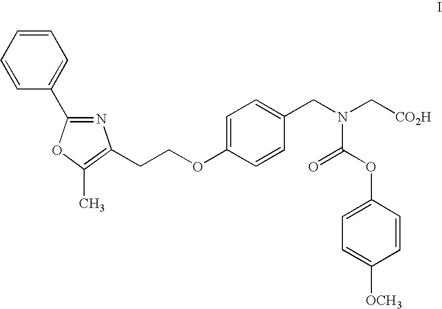

26. The crystalline form according to claim 25 comprising the N-1 form.

27. The crystalline form according to claim 25 characterized by one or more of the following:
a) unit cell parameters substantially equal to the following:
Cell dimensions
a=4.793(1) Å
b=19.914(4) Å
c=27.696(4) Å
α=90 degrees
β=94.52(1) degrees
γ=90 degrees
Space group P2₁/c
Molecules/asymmetric unit 1
wherein measurement of said crystalline form is at room temperature, and which is characterized by fractional atomic coordinates substantially as listed in Table 4;
b) a powder x-ray diffraction pattern comprising 2θ values (CuKα λ=1.5418 Å) selected from the group consisting of 6.4±0.1, 8.9±0.1, 11.0±0.1, 13.1±0.1, 13.6±0.1, 15.6±0.1, 19.1±0.1, 20.6±0.1, 22.1±0.1 and 23.0±0.1, at room temperature;
c) a solid state ¹³C NMR spectrum having substantially similar peak positions at 10.1, 23.8, 47.4, 50.4, 56.5, 67.4, 110.3 or 110.9, 118.0 or 119.8, 124.1, 126.1, 128.0, 129.0, 130.8, 131.1, 133.3, 144.0, 145.5, 155.8, 156.3, 158.6, 160.9 and 171.7 ppm, as determined on a 400 MHz spectrometer relative to TMS at zero;
d) a differential scanning calorimetry thermogram having a peak onset at about 140-144° C.;
e) thermal gravimetric analysis curve having less then 0.3% weight loss up to about 125° C.;
f) a moisture sorption isotherm having less then 0.3% moisture uptake in the range 25-75% RH at 25° C.; and/ or
g) a powder X-ray diffraction pattern substantially in accordance with that shown in FIG. 1.

28. A pharmaceutical composition comprising the crystalline form according to claim 25 and a pharmaceutically acceptable carrier or diluent.

29. A pharmaceutical composition comprising the crystalline form according to claim 25 in combination with one or more therapeutic agents selected from the group consisting of an antidiabetic agent, an anti-obesity agent, a anti-hypertensive agent, an anti-atherosclerotic agent and a lipid-lowering agent.

30. A method of treating diabetes, diabetic retinopathy, diabetic neuropathy, diabetic nephropathy, delayed wound healing, insulin resistance, hyperglycemia, hyperinsulinemia, elevated blood levels of fatty acids or glycerol, hyperlipidemia, hypertriglyceridemia, Syndrome X, or dyslipidemia in a mammal comprising administering to the mammal a therapeutically-effective amount of the crystalline form according to claim 25.
